(19) United States
(12) Patent Application Publication
(10) Pub. No.: US 2012/0321578 A1
(43) Pub. Date: Dec. 20, 2012

(54) SOLID WATER-IN-OIL EMULSION COMPRISING A VOLATILE HYDROCARBON SOLVENT, A POLYGLYCEROLATED SURFACTANT AND A POLAR WAX

(75) Inventors: Maitena Leuridan, Brie Comte Robert (FR); Agnes Themens, Bourg La Reine (FR); Christophe Dumousseaux, Antony (FR); Valerie Dique-Mouton, Chevilly Larue (FR)

(73) Assignee: L'OREAL, Paris (FR)

(21) Appl. No.: 13/515,536

(22) PCT Filed: Dec. 14, 2010
(86) PCT No.: PCT/EP10/69618

§ 371 Date: Jun. 18, 2012

Related U.S. Application Data
(60) Provisional application No. 61/290,942, filed on Dec. 30, 2009.

(30) Foreign Application Priority Data
Dec. 18, 2009 (FR) 09 59180

(51) Int. Cl. A61K 8/92; A61Q 19/00; A61K 8/98; A61Q 1/02; A61K 8/97

(52) U.S. Cl. 424/63; 514/772

(57) ABSTRACT

The invention relates to a solid water-in-oil emulsion comprising an aqueous phase emulsified in a fatty phase, comprising: one or more volatile linear alkane(s), especially C7-C14 alkane(s), a non-silicone polyglycerolated surfactant and a polar wax, especially a natural or natural-origin polar wax.

The present invention relates to a solid water-in-oil emulsion comprising a volatile hydrocarbon solvent, a non-silicone polyglycerolated surfactant and a polar wax, especially a natural or natural-origin polar wax, the emulsion being suitable for use in the cosmetics field. The invention also relates to a makeup or care method for keratin materials, more particularly the skin, comprising the application of the composition to said keratin materials.

According to the invention, keratin materials are the skin of the body and the mucosae, for example the face or the lips, and also the hair of the body and head, and especially the lashes.

The makeup composition according to the invention is more particularly a skin makeup composition, such as a foundation, eyeshadow, blusher, concealer, body makeup product or lipstick. The invention relates more specifically to a foundation composition.

The care composition may be a skincare product such as a care base for the skin, a care cream (day cream, night cream, anti-wrinkle cream) or a makeup base; a care composition for the lips (lip balm); a sun protection or self-tanning composition; or a deodorant.

Skin makeup products such as foundations are known in a very wide variety of formulations, including loose powder, compact powder, cast solid product, stick and fluid cream.

Cast solid products may be anhydrous or else in the form of emulsions. The emulsions generally contain fats such as oils and solid waxes, water and a particulate phase generally composed of fillers and pigments.

The solid emulsion does not flow under its own weight at ambient temperature, and is suitable for being packaged in a case: to apply the product, the user may take the product directly, breaking it down using the fingers or an applicator such as a sponge.

These solid emulsions are a category of foundation which is developing more and more, combining the ease of use of the compact products with the application of the cream products.

To produce this type of solid water-in-oil emulsion, all of the ingredients are brought to a high temperature to carry out emulsification. The emulsion solidifies in the course of cooling, to give a compact and solid appearance at ambient temperature. The emulsion must therefore not only contain ingredients which are resistant to temperature and do not give rise to safety problems, but also have good heat stability during manufacture and during the various subsequent casting operations.

The end product, in order to be sensorially acceptable after application, and to avoid any sensation of stickiness and weightiness, must also contain volatile oils.

In order to resolve all of these constraints, volatile silicone oils in combination with silicone surfactants are traditionally used. This combination, however, does not provide a fresh effect sufficient for application, and does not promote a sensation of moisturizing over the course of the day. Nor is it compatible with the search for cosmetic products which are formed wholly or partly of natural or natural-origin ingredients.

A natural compound is a compound obtained directly from the earth or the ground, or from plants or animals, by way, where appropriate, of one or more physical processes, such as grinding, refining, distilling, purifying or filtering, for example.

Natural-origin compounds are natural compounds which have undergone one or more auxiliary industrial or chemical treatments, producing modifications which do not adversely affect the essential qualities of these compounds, and/or are compounds comprising primarily natural ingredients, with or without having undergone transformations as indicated above.

Non-limitative examples of auxiliary industrial or chemical treatments which produce modifications that do not adversely affect the essential qualities of a natural compound include those authorized by monitoring organizations such as Ecocert (Standard for Biological and Ecological Cosmetic Products, January 2003) or those defined in manuals which are recognized in the art, such as Cosmetics and Toiletries Magazine, 2005, vol. 120, 9:10.

Absent from the market, therefore, is a solid emulsion providing a substantial sensation of freshness allied with finesse in application and a sensation of moisturizing.

The aim of the present invention, specifically, is to meet this need.

The inventors have found, unexpectedly, that the combination in a solid water-in-oil emulsion of
a) a volatile linear alkane, more particularly a C7-C14 alkane,
b) a non-silicone polyglycerolated surfactant and
c) a natural or natural-origin polar wax allows a product to be obtained which combines very good hot and cold stability with a fresh and moisturizing texture.

The invention accordingly provides in particular a solid water-in-oil emulsion comprising an aqueous phase emulsified in a fatty phase, comprising:
one or more volatile linear alkane(s), especially C7-C14 alkane(s),
a non-silicone polyglycerolated surfactant and
a polar wax, especially a natural or natural-origin polar wax.

A solid composition is a composition which does not flow under its own weight at ambient temperature (25° C.) within one hour.

The solid water-in-oil emulsion according to the invention may be coloured or non-coloured and may take the form of a foundation, eyeshadow, blusher, concealer, body makeup product or lipstick, a care base for the skin or a care cream, a care composition for the lips, a sun protection or self-tanning composition, or a deodorant.

More particularly it is a foundation.

The invention likewise provides a cosmetic makeup or non-therapeutic care method for keratin materials, more particularly the skin, comprising the application to said keratin material of a composition according to the invention.

### Volatile Hydrocarbon Solvent

The volatile hydrocarbon solvent used according to the invention comprises one or more volatile linear alkanes as defined hereinafter.

The volatile linear alkane or alkanes used according to the invention preferably have a flash point of more than 60° C. The flash point is measured more particularly in accordance with ISO Standard 3679.

The phrase “one or more volatile linear alkanes” is synonymous with “one or more volatile linear alkane oils”.

A volatile linear alkane suitable for the invention is liquid at ambient temperature (around 25° C.) and at atmospheric pressure (760 mmHg).

The expression “volatile linear alkane”, suitable for the invention, is understood to mean a cosmetic linear alkane capable of evaporating on contact with the skin in less than one hour, at ambient temperature (25° C.) and atmospheric pressure (760 mmHg, that is to say 101 325 Pa), that is liquid at ambient temperature, and that has, in particular, an evaporation rate ranging from 0.01 to 15 mg/cm²/min at ambient temperature (25° C.) and atmospheric pressure (760 mmHg).

Preferably, the “volatile linear alkanes” suitable for the invention have an evaporation rate ranging from 0.01 to 3.5 mg/cm²/min at ambient temperature (25° C.) and atmospheric pressure (760 mmHg).

Preferably, the “volatile linear alkanes” suitable for the invention have an evaporation rate ranging from 0.01 to 1.5 mg/cm²/min at ambient temperature (25° C.) and atmospheric pressure (760 mmHg).

More preferably, the “volatile linear alkanes” suitable for the invention have an evaporation rate ranging from 0.01 to 0.8 mg/cm²/min at ambient temperature (25° C.) and atmospheric pressure (760 mmHg).

More preferably, the “volatile linear alkanes” suitable for the invention have an evaporation rate ranging from 0.01 to 0.3 mg/cm²/min at ambient temperature (25° C.) and atmospheric pressure (760 mmHg).

More preferably, the “volatile linear alkanes” suitable for the invention have an evaporation rate ranging from 0.01 to 0.12 mg/cm²/min at ambient temperature (25° C.) and atmospheric pressure (760 mmHg).

The evaporation rate of a volatile alkane according to the invention (and more generally of a volatile solvent) may especially be evaluated by means of the protocol described in WO 06/013413, and more particularly by means of the protocol described below.

Introduced into a crystallizing dish (diameter: 7 cm), placed on a balance that is located in a chamber of around 0.3 m³, the temperature (25° C.) and hygrometry (50% relative humidity) of which are regulated, are 15 g of volatile hydrocarbon solvent.

The liquid is left to evaporate freely, without being stirred, ventilation being provided by a fan (PAPST-MOTOREN, reference 8550 N, rotating at 2700 rpm) placed in a vertical position above the crystallizing dish containing the volatile hydrocarbon solvent, the blades being directed towards the crystallizing dish, at a distance of 20 cm relative to the base of the crystallizing dish.

The mass of volatile hydrocarbon solvent remaining in the crystallizing dish is measured at regular time intervals.

The evaporation profile of the solvent is then obtained by plotting the curve of the amount of product evaporated (in mg/cm²) as a function of the time (in min).

Then the evaporation rate, which corresponds to the tangent at the origin of the curve obtained, is calculated. The evaporation rates are expressed as mg of volatile solvent evaporated per unit area (cm²) and per unit time (minutes).

According to one preferred embodiment, the “volatile linear alkanes” suitable for the invention have a non-zero vapour pressure (also known as saturation vapour pressure) at ambient temperature, in particular a vapour pressure ranging from 0.3 Pa to 6000 Pa.

Preferably, the “volatile linear alkanes” suitable for the invention have a vapour pressure ranging from 0.3 to 2000 Pa at ambient temperature (25° C.).

Preferably, the “volatile linear alkanes” suitable for the invention have a vapour pressure ranging from 0.3 to 1000 Pa at ambient temperature (25° C.).

More preferably, the “volatile linear alkanes” suitable for the invention have a vapour pressure ranging from 0.4 to 600 Pa at ambient temperature (25° C.).

Preferably, the “volatile linear alkanes” suitable for the invention have a vapour pressure ranging from 1 to 200 Pa at ambient temperature (25° C.).

More preferably, the “volatile linear alkanes” suitable for the invention have a vapour pressure ranging from 3 to 60 Pa at ambient temperature (25° C.).

According to one embodiment, a volatile linear alkane suitable for the invention may have a flash point in the range that varies from 30 to 120° C., and more particularly from 40 to 100° C. The flash point is, in particular, measured according to ISO Standard 3679.

According to one preferred embodiment, a volatile linear alkane is used that has a flash point of more than 60° C.

According to one embodiment, an alkane suitable for the invention may be a volatile linear alkane comprising from 7 to 14 carbon atoms.

Preferably, the “volatile linear alkanes” suitable for the invention comprise from 8 to 14 carbon atoms.

Preferably, the “volatile linear alkanes” suitable for the invention comprise from 9 to 14 carbon atoms.

Preferably, the “volatile linear alkanes” suitable for the invention comprise from 10 to 14 carbon atoms.

Preferably, the “volatile linear alkanes” suitable for the invention comprise from 11 to 14 carbon atoms.

According to one advantageous embodiment, the “volatile linear alkanes” suitable for the invention have an evaporation rate, as defined above, ranging from 0.01 to 3.5 mg/cm²/min at ambient temperature (25° C.) and atmospheric pressure (760 mmHg) and comprise from 8 to 14 carbon atoms.

A volatile linear alkane suitable for the invention may advantageously be of plant origin.

Preferably, the volatile linear alkane or the mixture of volatile linear alkanes present in the composition according to the invention comprises at least one ¹⁴C isotope of carbon (carbon-14), in particular the ¹⁴C isotope may be present in a ¹⁴C/¹²C ratio greater than or equal to 1×10⁻¹⁶, preferably greater than or equal to 1×10⁻¹⁵, more preferably greater than or equal to 7.5×10⁻¹⁴, and better still greater than or equal to 1.5×10⁻¹³. Preferably, the ¹⁴C/¹²C ratio ranges from 6×10⁻¹³ to 1.2×10⁻¹².

The amount of ¹⁴C isotopes in the volatile linear alkane or the mixture of volatile linear alkanes may be determined by methods known to a person skilled in the art such as the Libby counting method, liquid scintillation spectrometry or else accelerator mass spectrometry.

Such an alkane may be obtained, directly or in several steps, from a plant raw material such as an oil, a butter, a wax, etc.

As examples of alkanes suitable for the invention, mention may be made of those described in patent application WO 2007/068371 or WO 2008/155059 by Cognis (mixtures of different alkanes that differ by at least one carbon). These alkanes are obtained from fatty alcohols that are themselves obtained from coconut oil or palm oil.

By way of example of linear alkanes suitable for the invention, mention may be made of n-heptane (C7), n-octane (C8), n-nonane (C9), n-decane (010), n-undecane (C11), n-dodecane (C12), n-tridecane (C13), n-tetradecane (C14) and mixtures thereof. According to one particular embodiment, the volatile linear alkane is chosen from n-nonane, n-undecane, n-dodecane, n-tridecane, n-tetradecane, and mixtures thereof.

According to one preferred embodiment, mention may be made of the mixtures of n-undecane (C11) and of n-tridecane (C13) obtained in examples 1 and 2 of application WO 2008/155059 by Cognis.

Mention may also be made of n-dodecane (C12) and n-tetradecane (C14) sold by Sasol respectively under the references PARAFOL 12-97 and PARAFOL 14-97, and also mixtures thereof.

The volatile linear alkane could be used alone.

Alternatively or preferably a mixture of at least two different volatile linear alkanes could be used, that differ from one another by a carbon number n of at least 1, in particular that differ from one another by a carbon number of 1 or of 2.

According to a first embodiment, use is made of a mixture of at least two different volatile linear alkanes comprising from 10 to 14 carbon atoms that differ from one another by a carbon number of at least 1. By way of examples, mention may especially be made of the C10/C11, C11/C12 or C12/C13 mixtures of volatile linear alkanes.

According to another embodiment, use is made of a mixture of at least two different volatile linear alkanes comprising from 10 to 14 carbon atoms that differ from one another by a carbon number of at least 2. By way of examples, mention may especially be made of the C10/C12 or C12/C14 mixtures of volatile linear alkanes for an even carbon number n and the C11/C13 mixture for an odd carbon number n.

According to one preferred embodiment, use is made of a mixture of at least two different volatile linear alkanes comprising from 10 to 14 carbon atoms that differ from one another by a carbon number of at least 2, and in particular a C11/C13 mixture of volatile linear alkanes or a C12/C14 mixture of volatile linear alkanes.

Other mixtures combining more than 2 volatile linear alkanes according to the invention, such as for example a mixture of at least 3 different volatile linear alkanes comprising from 7 to 14 carbon atoms that differ from one another by a carbon number of at least 1, are also part of the invention, but the mixtures of 2 volatile linear alkanes according to the invention are preferred (binary mixtures), said 2 volatile linear alkanes preferably representing more than 95% and better still more than 99% by weight of the total content of volatile linear alkanes in the mixture. According to one particular embodiment of the invention, in a mixture of volatile linear alkanes, the volatile linear alkane having the smallest carbon number is predominant in the mixture.

According to another embodiment of the invention, use is made of a mixture of volatile linear alkanes in which the volatile linear alkane having the highest carbon number is predominant in the mixture.

By way of examples of mixtures suitable for the invention, mention may especially be made of the following mixtures:
from 50% to 90% by weight, preferably from 55% to 80% by weight, more preferably from 60% to 75% by weight of Cn volatile linear alkane with n ranging from 7 to 14,
from 10% to 50% by weight, preferably from 20% to 45% by weight, more preferably from 24% to 40% by weight of Cn+x volatile linear alkane with x greater than or equal to 1, preferably x=1 or x=2, with n+x between 8 and 14,

relative to the total weight of the alkanes in said mixture.

In particular, said mixture of alkanes according to the invention contains:
less than 2% by weight, preferably less than 1% by weight, of branched hydrocarbons;
and/or less than 2% by weight, preferably less than 1% by weight, of aromatic hydrocarbons;
and/or less than 2% by weight, preferably less than 1% by weight and preferentially less than 0.1% by weight of unsaturated hydrocarbons in the mixture.

More particularly, a volatile linear alkane suitable for the invention may be used in the form of an n-undecane/n-tridecane mixture.

In particular, use will be made of a mixture of volatile linear alkanes comprising:
from 55% to 80% by weight, preferably from 60% to 75% by weight, of C11 volatile linear alkane (n-undecane),
from 20% to 45% by weight, preferably from 24% to 40% by weight, of C13 volatile linear alkane (n-tridecane),

relative to the total weight of the alkanes in said mixture.

According to one particular embodiment, the mixture of alkanes is an n-undecane/n-tridecane mixture. In particular, such a mixture may be obtained according to example 1 or example 2 of WO 2008/155059.

According to another particular embodiment, use is made of the n-dodecane sold under the reference PARAFOL 12-97 by Sasol.

According to another particular embodiment, use is made of the n-tetradecane sold under the reference PARAFOL 14-97 by Sasol.

According to yet another embodiment, use is made of a mixture of n-dodecane and of n-tetradecane.

The volatile linear alkane or alkanes are present in the composition preferably in an amount of from 0.5% to 25%, preferably from 1% to 18%, more preferably from 3% to 12% by weight, relative to the total weight of said composition. Too low a content may lead to a texture which is heavy and sticky after application. Too high a content may give rise to problems in terms of recrystallization of the bulk product in the casting operation (strong compatibility between this solvent and the wax crystals).

According to one particular embodiment, the composition of the invention may comprise at least 50% by weight of volatile linear alkane(s) relative to the total amount of volatile oil(s) in the composition. More particularly, a composition of the invention may comprise at least 60%, more particularly at least 70% and more particularly still at least 80%, at least 90% or 100% of volatile linear alkane(s), relative to the total amount of volatile oil(s) in the composition.

A composition of the invention comprising 100% of volatile linear alkane(s) relative to the total amount of volatile oil(s) comprises a volatile oily phase composed exclusively of volatile linear alkane(s).

According to one particular embodiment of the invention, the composition may comprise less than 10% by weight, or even less than 5% by weight, or even less than 2% by weight, or may even be devoid, of cyclic silicone oil.

According to another particular embodiment of the invention, the composition may comprise less than 10% by weight, or even less than 5% by weight, or even less than 2% by weight, or may even be devoid, of silicone oil.

### Non-Silicone Polyglycerolated Surfactant

The solid water-in-oil emulsion according to the invention comprises at least one non-silicone polyglycerolated surfactant.

The polyglycerolated surfactant or surfactants are preferably selected from the compounds of the following formula RO[CH₂CH(CH₂OH)O]ₘH, RO[CH₂CH(OH)CH₂O]ₘH or RO[CH(CH₂OH)CH₂O]ₘH, in which R represents a linear or branched, saturated or unsaturated hydrocarbon radical containing from 8 to 40 carbon atoms, preferably from 10 to 30 carbon atoms and more preferably from 10 to 20 carbon atoms and m is an integer between 1 and 30, preferably between 1 and 10 and more particularly from 1.5 to 6.

R may optionally comprise heteroatoms such as, for example, oxygen and nitrogen. More particularly, R may optionally comprise one or more hydroxyl and/or ether and/or amide groups.

R preferably denotes a C₁₀-C₂₀ alkyl or alkylene radical which is optionally mono- or polyhydroxylated. According to one particular embodiment, R is an alkyl group and m is an integer between 1 and 10.

Non-silicone polyglycerolated surfactants of these kinds are available commercially in the ranges Sunsoft from Taiyo Kagaku, Tegocare and Tegosoft from Evonik-Degussa, Dehymuls and Lameform from Cognis, and Arlacel from Croda.

Mention may be made especially of the following non-silicone polyglycerolated surfactants, in order of increasing HLB:
polyglyceryl-2 distearate, such as that sold under the name Emalex PGSA by Nihom Emulsion,
polyglyceryl-10 decastearate, such as that sold under the name Sunsoft Q-1810S by TAIYO KAGAKU,
polyglyceryl-3 ricinoleate (and) sorbitan isostearate, such as that sold under the name Arlacel 1690 by CRODA,
polyglyceryl-5 hexastearate, such as that sold under the name Sunsoft A-186E by TAIYO KAGAKU,
polyglyceryl-10 pentaoleate, such as that sold under the name Sunsoft Q-175S by TAIYO KAGAKU,
polyglyceryl-10 pentastearate, such as that sold under the name Sunsoft Q-185S by TAIYO KAGAKU,
polyglyceryl-4 isostearate, such as that sold under the name Isolan GI 34 by EVONIK GOLDSCHMIDT,
polyglyceryl-4 diisostearate polyhydroxy-stearate sebacate, such as that sold under the name Isolan GPS by EVONIK GOLDSCHMIDT,
polyglyceryl-2 dipolyhydroxystearate, such as that sold under the name Dehymuls PGPH by COGNIS,
polyglyceryl-3 diisostearate, such as that sold under the name Lameform TGI by COGNIS,
polyglyceryl-5 trioleate, such as that sold under the name Sunsoft A-173E by TAIYO KAGAKU,
polyglyceryl-2 oleate, such as that sold under the name Sunsoft Q-17B by TAIYO KAGAKU,
polyglyceryl-2 caprylate, such as that sold under the name Sunsoft Q-81B by TAIYO KAGAKU,
polyglyceryl-2 laurate, such as that sold under the name Sunsoft Q-12D by TAIYO KAGAKU,
polyglyceryl-3 polyricinoleate, such as that sold under the name Crester PR by CRODA,

and mixtures thereof.

The non-silicone polyglycerolated surfactants suitable for the invention have an HLB of between 1 and 14, preferably between 3 and 10.

An “HLB of between 1 and 14” is understood to refer to a surfactant possessing at 25° C. an HLB (hydrophilic-lipophilic balance) in the sense of Griffin of between 1 and 14.

The Griffin HLB value is defined in J. Soc. Cosm. Chem. 1954 (volume 5), pages 249-256.

Reference may be made to the Encyclopaedia of Chemical Technology, KIRK-OTHMER, volume 22, pp. 333-432, 3^{rd} edition, 1979, WILEY, as regards the definition of the properties and functions (emulsifying) of surfactants.

According to one preferred embodiment, the non-silicone polyglycerolated surfactant is selected from polyglyceryl-4 isostearate, polyglyceryl-3 diisostearate and a mixture thereof.

Preference will be given to using polyglyceryl-3 diisostearate, such as that sold under the name Lameform TGI by Cognis, polyglyceryl-4 isostearate, such as the product sold under the name Isolan GI 34 by Goldschmidt, or a mixture thereof.

According to one particularly preferred embodiment, use will be made of polyglyceryl-3 diisostearate, such as that sold under the name Lameform TGI by Cognis.

The non-silicone polyglycerolated surfactant is preferably present in the composition at a concentration of from 0.5% to 6%, preferably from 2% to 5%. Too low a concentration may not be beneficial for the stability of the emulsion, while too high a concentration may give rise to a heavy and sticky texture.

### Polar Wax

A solid water-in-oil emulsion according to the invention comprises at least one polar wax, preferably at least one natural or natural-origin polar wax.

A natural compound is a compound obtained directly from the earth or the ground, or from plants or animals, by way, where appropriate, of one or more physical processes, such as grinding, refining, distilling, purifying or filtering, for example.

Natural-origin compounds are natural compounds which have undergone one or more auxiliary industrial or chemical treatments, producing modifications which do not adversely affect the essential qualities of these compounds, and/or are compounds comprising primarily natural ingredients, with or without having undergone transformations as indicated above.

A wax in the sense of the present invention is a lipophilic fatty compound which is solid at ambient temperature (25° C.) and exhibits a reversible solid/liquid state change, having a melting temperature of more than 30° C. and possibly up to 200° C. and a hardness of more than 0.5 MPa, and exhibiting, in the solid state, an anisotropic crystalline organization. By taking the wax to its melting temperature, it is possible to make it miscible with oils and to form a microscopically homogeneous mixture, but, by taking the temperature of the mixture to the ambient temperature, recrystallization of the wax from the oils of the mixture takes place.

The hardness of the wax is determined by measuring the compressive force at 20° C. by means of the texturometer sold under the name TA-XT2i by Rheo, which is equipped with a stainless steel cylinder 2 mm in diameter that moves at a measuring speed of 0.1 mm/s and penetrates the wax to a depth of 0.3 mm. For carrying out the hardness measurement, the wax is melted at a temperature equal to the melting point of the wax +20° C. The melted wax is poured into a container with a diameter of 30 mm and a depth of 20 mm. The wax is recrystallized at ambient temperature (25° C.) for 24 hours, and then stored at 20° C. for at least one hour before the hardness measurement is performed. The value of the hardness is the compressive force measured, divided by the surface area of the cylinder of the texturometer in contact with the wax.

A polar wax in the sense of the present invention is a wax whose solubility parameter δₐ at 25° C. is other than 0 (J/cm³)^{1/2}.

More particularly, a polar wax is a wax whose chemical structure is essentially formed, or even composed, of atoms of carbon and hydrogen, and includes at least one highly electronegative heteroatom such as an oxygen, nitrogen or phosphorus atom.

The definition and calculation of solubility parameters in the Hansen three-dimensional solubility space are described in the article by C. M. HANSEN: “The three dimensional solubility parameters”, J. Paint Technol. 39, 105 (1967).

According to this Hansen space:
δ_{D} characterizes the London dispersion forces resulting from the formation of dipoles induced on molecular impacts;
δₚ characterizes the Debye interaction forces between permanent dipoles and also the Keesom interaction forces between induced dipoles and permanent dipoles;
δₕ characterizes the specific interaction forces (hydrogen bonds, acid/base, donor/acceptor, etc.);
δₕ is determined by the following equation:

δₐ=(δₚ²+δₕ²)^{1/2}

The parameters δₚ, δₕ, δ_{D} and δₐ are expressed in (J/cm³)^{1/2}.

The polar waxes which can be used in the compositions according to the invention are selected from waxes which are solid at ambient temperature and are of animal, vegetable, mineral and/or synthetic origin, and mixtures thereof.

The polar wax or waxes are preferably hydrocarbon waxes.

A hydrocarbon wax is a wax essentially formed, or even composed, of atoms of carbon and hydrogen, and optionally atoms of oxygen and nitrogen, but containing no silicon or fluorine atom. Such a wax may contain alcohol, ester, ether, carboxylic acid, amine and/or amide groups.

Illustrative instances of natural or natural-origin polar waxes suitable for the invention may include beeswax, lanolin wax, rice bran wax, carnauba wax, candelilla wax, shellac wax, montan wax, orange wax, lemon wax, laurel wax, hydrogenated jojoba oil, olive wax and mixtures thereof.

According to one particular embodiment, use will be made of candelilla wax, more particularly a purified candelilla wax, such as that sold under the name NC1630 by Cera Rica Noda.

The polar wax or waxes are present in the composition preferably at a concentration of from 0.5% to 10%, preferably from 2% to 6%, so as to give a texture which is both solid and creamy on application.

According to one preferred embodiment, the solid water-in-oil emulsion according to the invention is characterized in that:
a) the volatile linear alkane or alkanes are present in an amount of from 0.5% to 25% by weight, preferably from 1% to 18% and more preferably from 3% to 12% by weight relative to the total weight of said composition,
b) the non-silicone polyglycerolated surfactant is present in the composition in an amount of from 0.5% to 6%, preferably from 2% to 5% by weight relative to the total weight of said composition, and
c) wherein the natural or natural-origin polar wax is present in the composition in an amount of from 0.5% to 10% by weight, preferably from 2% to 6% by weight, relative to the total weight of said composition.

### Physiologically Acceptable Medium

Further to the compounds indicated above, a composition according to the invention comprises a physiologically acceptable medium.

A physiologically acceptable medium is a medium which is suitable particularly for application of a composition of the invention to keratin materials, especially the skin and the lips.

The physiologically acceptable medium is generally suited to the nature of the substrate to which the composition is to be applied, and to the form in which the composition is to be presented.

### Aqueous Phase

The aqueous phase of the solid emulsion according to the invention generally comprises water. The water may be a floral water such as cornflower water and/or a mineral water such as Vittel water, Lucas water or La Roche Posay water, and/or a spring water. The water may be present in the emulsion according to the invention in an amount of from 10% to 40% by weight, relative to the total weight of the composition, preferably of from 15% to 30% by weight, and preferentially of from 15% to 25% by weight.

The aqueous phase may also comprise water-miscible solvents (at ambient temperature—25° C.) such as, for example,

monoalcohols having from 2 to 6 carbon atoms, such as ethanol and isopropanol;

polyols having especially from 2 to 20 carbon atoms, preferably having from 2 to 10 carbon atoms, and preferentially having from 2 to 6 carbon atoms, such as glycerol, propylene glycol, butylene glycol, pentylene glycol, hexylene glycol, dipropylene glycol and diethylene glycol;

glycol ethers (having especially from 3 to 16 carbon atoms) such as mono-, di- or tripropylene glycol alkyl (C₁-C₄) ethers and mono-, di- or triethylene glycol alkyl (C₁-C₄) ethers;

and mixtures thereof.

The emulsion according to the invention may comprise a water-miscible organic solvent, especially a polyol, in an amount of from 1% to 20% by weight, relative to the total weight of the composition, and preferably of from 3% to 15% by weight.

The aqueous phase may further comprise stabilizers, for example sodium chloride, magnesium dichloride and magnesium sulphate.

The aqueous phase may also comprise any water-soluble or water-dispersible compound which is compatible with an aqueous phase, such as gellants, film-forming polymers, thickeners, surfactants and mixtures thereof.

The aqueous phase preferably may be present in the emulsion according to the invention in an amount of from 15% to 50% by weight, preferably of from 25% to 45% by weight, relative to the total weight of the emulsion, and preferentially of from 25% to 35% by weight.

According to one preferred embodiment, the composition of the invention comprises a polyol in the aqueous phase in order to promote moisturizing.

### Liquid Fatty Phase

Further to the above-described volatile hydrocarbon solvent having a flash point of more than 60° C., the composition according to the invention may further comprise additional oils, which are preferably non-silicone oils, and are volatile or non-volatile and fluorinated or non-fluorinated.

An oil is any fatty substance which is in liquid form at ambient temperature (20-25° C.) and at atmospheric pressure.

It may be animal, plant, mineral or synthetic in origin. According to one embodiment, oils of plant origin are preferred.

For the purposes of the present invention, a “volatile oil” is an oil (or non-aqueous medium) which is capable of evaporating on contact with the skin in less than one hour at ambient temperature and at atmospheric pressure. The volatile oil is a volatile cosmetic oil which is liquid at ambient temperature and has, especially, a non-zero vapour pressure at ambient temperature and at atmospheric pressure, more particularly having a vapour pressure of from 0.13 Pa to 40 000 Pa (10⁻³ to 300 mmHg), and preferably of from 1.3 Pa to 13 000 Pa (0.01 to 100 mmHg), and preferentially of from 1.3 Pa to 1300 Pa (0.01 to 10 mmHg).

For the purposes of the present invention, a “non-volatile oil” is an oil having a vapour pressure of less than 0.13 Pa.

For the purposes of the present invention, a “silicone oil” is an oil comprising at least one silicon atom, and especially at least one Si—O group.

A “fluorinated oil” is an oil comprising at least one fluorine atom.

A “hydrocarbon oil” is an oil containing primarily hydrogen and carbon atoms.

The oils may optionally comprise oxygen, nitrogen, sulphur and/or phosphorus atoms, in the form of hydroxyl or acid radicals, for example.

### Volatile Oils

The volatile oils may be selected from hydrocarbon oils having from 8 to 16 carbon atoms, and especially C₈-C₁₆ branched alkanes (also called isoparaffins), such as isododecane (also called 2,2,4,4,6-pentamethyl-heptane), isodecane, isohexadecane, and, for example, the oils sold under the trade names ISOPARS® or PERMETHYLS®.

### Non-Volatile Oils

The non-volatile oils may in particular be selected from non-volatile fluorinated hydrocarbon oils.

As non-volatile hydrocarbon oil, mention may in particular be made of:

hydrocarbon oils of animal origin,

hydrocarbon oils of plant origin, such as phytostearyl esters, for instance phytostearyl oleate, phytostearyl isostearate and lauroyl/octyldodecyl/phytostearyl glutamate (Ajinomoto, Eldew PS203), triglycerides constituted of fatty acid esters and of glycerol, in particular in which the fatty acids may have chain lengths ranging from C₄ to C₃₆, and in particular from C₁₈ to C₃₆, it being possible for these oils to be linear or branched, and saturated or unsaturated; these oils may in particular be heptanoic or octanoic triglycerides, shea oil, alfalfa oil, poppyseed oil, millet oil, barley oil, rye oil, candlenut oil, passionflower oil, shea butter, aloe oil, sweet almond oil, peach kernel oil, groundnut oil, argan oil, avocado oil, baobab oil, borage oil, broccoli oil, calendula oil, camelina oil, canola oil, carrot oil, safflower oil, hemp oil, rapeseed oil, cottonseed oil, coconut oil, marrow seed oil, wheat germ oil, jojoba oil, lily oil, macadamia oil, maize oil, meadowfoam oil, St. John's wort oil, monoi oil, hazelnut oil, apricot kernel oil, nut oil, olive oil, evening primrose oil, palm oil, blackcurrant seed oil, kiwi seed oil, grapeseed oil, pistachio oil, pumpkin oil, winter squash oil, quinoa oil, musk rose oil, sesame oil, soya bean oil, sunflower oil, castor oil and watermelon oil, and mixtures thereof, or alternatively caprylic/capric acid triglycerides, for instance those sold by the company Stéarineries Dubois or those sold under the names Miglyol 810®, 812® and 818® by the company Dynamit Nobel,

synthetic ethers having from 10 to 40 carbon atoms, such as dicapryl ether,

synthetic esters, for instance oils of formula R₁COOR₂, in which R₁ represents a linear or branched fatty acid residue containing from 1 to 40 carbon atoms and R₂ represents a hydrocarbon chain, in particular a branched chain, containing from 1 to 40 carbon atoms provided that R₁+R₂ is ≧10. The esters may in particular be selected from fatty acid and alcohol esters, such as, for example, cetostearyl octanoate, isopropyl alcohol esters, such as isopropyl myristate or isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, isononanoic acid esters, for instance isononyl isononanoate, isotridecyl isononanoate,

polyol esters and pentaerythritol esters, for instance dipentaerythrityl tetrahydroxy-stearate/tetraisostearate,

fatty alcohols that are liquid at ambient temperature, with a branched and/or unsaturated carbon chain containing from 12 to 26 carbon atoms, for instance 2-octyldodecanol, isostearyl alcohol, oleyl alcohol,

C₁₂-C₂₂ higher fatty acids, such as oleic acid, linoleic acid or linolenic acid, and mixtures thereof,

dialkyl carbonates, the two alkyl chains possibly being identical or different, such as dicaprylyl carbonate sold under the name Cetiol CC® by Cognis, and

oils of high molar mass, having in particular a molar mass ranging from approximately 400 to approximately 2000 g/mol, in particular from approximately 650 to approximately 1600 g/mol. As oils of high molar mass that can be used in the present invention, mention may in particular be made of esters of linear fatty acids having a total carbon number ranging from 35 to 70, such as pentaerythrityl tetrapelargonate, hydroxylated esters, such as polyglyceryl-2 triisostearate, aromatic esters, such as tridecyl trimellitate, esters of C₂₄-C₂₈ branched fatty acids or fatty alcohols, such as those described in U.S. Pat. No. 6,491,927, and pentaerythritol esters, and especially triisoarachidyl citrate, glyceryl triisostearate, glyceryl 2-tridecyl tetradecanoate, polyglyceryl-2 tetraisostearate or else pentaerythrityl 2-tetradecyl tetradecanoate, and also mixtures of these various oils.

According to one embodiment, a composition of the invention may advantageously comprise less than 10% by weight, or even less than 5% by weight, or even less than 2% by weight, relative to the total weight of the composition, or may even be devoid, of silicone oil, more particularly of cyclic silicone oil, and/or of mineral oil, and/or of branched volatile alkanes not directly obtained from plants or of plant origin, such as isododecane or isoparaffins.

### Pasty Compounds

A composition according to the invention may comprise at least one pasty compound. The presence of a pasty compound may have the advantageous effect of imparting enhanced comfort when a composition of the invention is applied to the keratin materials.

A compound of this kind may be advantageously selected from the following:
lanolin and its derivatives,
polymeric or non-polymeric fluoro compounds,
vinyl polymers, especially:
   olefin homopolymers,
   olefin copolymers,
   hydrogenated diene homopolymers and copolymers,
   linear or branched oligomers, homopolymers or copolymers of alkyl (meth)acrylates having preferably a C₈-C₃₀ alkyl group,
   oligomers, homopolymers and copolymers of vinyl esters having C₈-C₃₀ alkyl groups,
   oligomers, homopolymers and copolymers of vinyl ethers having C₈-C₃₀ alkyl groups,
fat-soluble polyethers resulting from the polyetherification of one or more C₂-C₁₀₀, more particularly C₂-C₅₀, diols,
fatty alcohol or acid esters,
and mixtures thereof.

The esters may include more particularly:
esters of an oligomeric glycerol, especially diglycerol esters, more particularly the condensates of adipic acid and glycerol, wherein some of the hydroxyl groups of the glycerols have reacted with a mixture of fatty acids, such as stearic acid, capric acid, stearic acid and isostearic acid and 12-hydroxystearic acid, of the kind more particularly of those sold under the brand name Softisan 649 by the company Sasol, or such as bisdiglyceryl polyacyladipate-2,
arachidyl propionate sold under the brand name Waxenol 801 by Alzo,
phytosterol esters,
triglycerides of fatty acids and derivatives thereof, such as hydrogenated cocoglycerides,
non-crosslinked polyesters resulting from the polycondensation of a linear or branched C₄-C₅₀ dicarboxylic or polycarboxylic acid with a C₂-C₅₀ diol or polyol,
aliphatic esters resulting from the esterification of an aliphatic hydroxycarboxylic acid ester with an aliphatic carboxylic acid,
and mixtures thereof.

According to one embodiment, a composition of the invention may advantageously comprise less than 10% by weight of fluoro and/or silicone pasty compounds, or even less than 5% by weight, or even less than 2% by weight of fluoro and/or silicone pasty compounds, relative to the total weight of the composition, or may even be devoid of fluoro and/or silicone pasty compounds.

### Gellants

According to the fluidity of the composition it is desired to obtain, one or more gellants may be incorporated into a composition of the invention.

A gellant suitable for the invention may be lipophilic. A lipophilic gellant may be organic or inorganic.

Lipophilic gellants may include, for example, modified clays such as modified magnesium silicate (bentone gel VS38 from Rheox), hectorite modified with distearyldimethylammonium chloride (CTFA name: Disteardimonium hectorite), sold under the name Bentone 38 CE by the company Rheox.

Inorganic lipophilic gellants may include optionally modified clays, such as hectorites modified with a C₁₀ to C₂₂ fatty acid ammonium chloride, such as hectorite modified with distearyldimethylammonium chloride, such as, for example, that sold under the name Bentone 38V® by Elementis.

Polymeric organic lipophilic gellants are, for example, the partly or totally crosslinked elastomeric organopolysiloxanes of three-dimensional structure, such as those sold under the names KSG6®, KSG16® and KSG18® by the company Shin Etsu, Trefil E-505C® and Trefil E-506C® by the company Dow Corning, Gransil SR-CYC®, SR DMF10®, SR-DC556®, SR 5CYC Gel®, SR DMF 10 Gel® and SR DC 556 Gel® by the company Grant Industries, and SF 1204® and JK 113® by the company General Electric; block copolymers of diblock, triblock or radial type, of the polystyrene/polyisoprene or polystyrene/poly-butadiene type, such as those sold under the name Luvitol HSB® by the company BASF, or of the polystyrene/copoly(ethylene-propylene) type, such as those sold under the name Kraton® by the company Shell Chemical Co., or else of the polystyrene/copoly(ethylene-butylene) type, blends of triblock and radial (star) copolymers in isododecane, such as those sold by the company Penreco under the name Versagel®, such as, for example, the blend of butylene/ethylene/styrene triblock copolymer and ethylene/propylene/styrene star copolymer in iso-dodecane (Versagel M 5960).

Lipophilic gellants which can be used in a cosmetic composition of the invention may further include esters of dextrin and fatty acid, such as dextrin palmitates, especially those sold under the name Rheopearl TL®, Rheopearl TL2-OR® or Rheopearl KL® by Chiba Flour.

Lipophilic gellants suitable for the invention may also include hydrogenated vegetable oils, such as hydrogenated castor oil.

Lipophilic gellants also suitable for the invention may include fatty alcohols, especially C₈ to C₂₆ alcohols, and more particularly C₁₂ to C₂₂ alcohols.

According to one embodiment, a fatty alcohol suitable for the invention may be selected from myristyl alcohol, cetyl alcohol, stearyl alcohol and behenyl alcohol.

Lipophilic gellants likewise suitable for the invention may include esters of fatty acid and glycerols, such as glyceryl stearate.

According to one embodiment, a composition of the invention may comprise at least one lipophilic gellant selected in particular from modified hectorites.

According to one embodiment, a composition of the invention may advantageously comprise less than 10% by weight of silicone gellants, or even less than 5%, or even less than 2% by weight of silicone gellants, relative to the total weight of the composition, or may even be devoid of silicone gellants.

### Fillers

A composition in accordance with the invention may also comprise at least one filler, of organic or inorganic nature.

The term “filler” should be understood to mean colourless or white solid particles of any form, which are in a form that is insoluble and dispersed in the medium of the composition. Being inorganic or organic in nature, they make it possible to endow the composition with softness, mattness and uniformity to the makeup.

The fillers used in the compositions according to the present invention may be of lamellar, globular or spherical form, in the form of fibres or in any other form intermediate between these defined forms.

The fillers according to the invention may or may not be surface-coated, and in particular they may be surface-treated with silicones, amino acids, fluoro derivatives or any other substance that promotes the dispersion and compatibility of the filler in the composition.

Among the inorganic fillers that can be used in the compositions according to the invention, mention may be made of talc, mica, silica, trimethyl siloxysilicate, kaolin, bentone, calcium carbonate, magnesium hydrogen carbonate, hydroxyapatite, boron nitride, hollow silica microspheres (Silica Beads from Maprecos), glass or ceramic microcapsules, silica-based fillers such as Aerosil 200, Aerosil 300; Sunsphere H-33, Sunsphere H-51 sold by Asahi Glass; Chemicelen sold by Asahi Chemical; composites of silica and of titanium dioxide, such as the TSG series sold by Nippon Sheet Glass, and mixtures thereof.

According to one preferred embodiment, a lamellar inorganic filler of the mica, sericite or talc type will be used, present preferably in an amount of from 0.5% to 20% by weight, relative to the total weight of the composition, preferably of from 2% to 15% by weight.

According to one particular embodiment, the composition according to the invention comprises at least one organic filler, more particularly a porous organic filler.

Among the organic fillers that can be used in the compositions according to the invention, mention may be made of polyamide powders (Nylon® Orgasol from Atochem), polymethyl methacrylate (PMMA) particles, poly-b-alanine powder and polyethylene powder, polytetrafluoroethylene powders (Teflon®), lauroyllysine, starch, tetrafluoroethylene polymer powders, hollow polymer microspheres such as Expancel (Nobel Industrie), Polypore® L 200 (Chemdal Corporation), silicone resin microbeads (for example, Tospearl® from Toshiba), polyurethane powders, in particular powders of crosslinked polyurethane comprising a copolymer, said copolymer comprising trimethylol hexyllactone, such as the polymer of hexamethylene diisocyanate/trimethylol hexyllactone, sold under the name Plastic Powder D-400® or Plastic Powder D-800® by the company Toshiki, carnauba microwaxes, such as that sold under the name MicroCare 350® by Micro Powders, synthetic wax microwaxes, such as that sold under the name MicroEase 114S® by Micro Powders, and mixtures thereof.

According to one preferred embodiment, the composition according to the invention contains at least one porous organic filler, for example Nylon powder or PMMA powder, present preferably in an amount of from 0.5% to 10% by weight, relative to the total weight of the composition, preferably of from 0.5% to 7% by weight.

Preference will be given to combining a lamellar inorganic filler with a porous organic filler, preferably in a weight ratio of lamellar inorganic filler to porous organic filler of from 0.5 to 10, preferably from 1 to 5.

The fillers may be present in the composition in an amount of from 0.1% to 25% by weight, relative to the total weight of the composition, preferably of from 1% to 20% by weight, and preferentially of from 5% to 15% by weight.

According to one preferred embodiment, the composition according to the invention comprises at least one colorant.

### Colorants

The emulsion according to the invention may comprise at least one colorant, which may be selected from water-soluble or fat-soluble dyes, pigments, nacres and mixtures thereof.

Pigments are white or coloured, organic or inorganic particles which are insoluble in the liquid organic phase and are intended for colouring and/or opacifying the composition.

Nacres are iridescent particles, produced in particular by certain molluscs in their shell or else synthesized, which are insoluble in the medium of the composition.

Dyes are compounds, generally organic, which are soluble in fats such as oils or in an aqueous-alcoholic phase.

The pigments may be organic or inorganic pigments. Pigments that may be used include metal oxides, for instance iron oxides (especially yellow, red, brown and black colour varieties), titanium dioxides, cerium oxide, zirconium oxide, chromium oxide; manganese violet, ultramarine blue, Prussian blue, ultramarine blue, ferric blue, bismuth oxychloride, nacre, mica coated with titanium oxide or with bismuth oxychloride, coloured nacreous pigments such as titanium mica with iron oxides, titanium mica with, in particular, ferric blue or chromium oxide, titanium mica with an organic pigment of the aforementioned type, and also nacreous pigments based on bismuth oxychloride, and mixtures thereof.

Preference is given to using iron oxide or titanium dioxide pigments.

The pigments are advantageously treated with a hydrophobic agent to make them compatible with the organic phase of the composition. The hydrophobic treatment agent may be selected from silicones such as methicones, dimethicones and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkyl-silazanes, polyhexafluoropropylene oxides, polyorgano-siloxanes containing perfluoroalkyl perfluoropolyether groups, amino acids; N-acylamino acids or their salts; lecithin, isopropyl triisostearyltitanate, and mixtures thereof.

The N-acylamino acids may comprise an acyl group having from 8 to 22 carbon atoms, such as for example a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

The alkyl term referred to in the compounds identified above denotes more particularly an alkyl group having from 1 to 30 carbon atoms, preferably having from 5 to 16 carbon atoms.

Hydrophobic treated pigments are described more particularly in patent application EP-A-1086683.

The fat-soluble dyes are, for example, Sudan red, D&C red No. 17, D&C green No. 6, β-carotene, soya oil, Sudan brown, D&C yellow No. 11, D&C violet No. 2, D&C orange No. 5, quinoline yellow, annatto and bromo acids.

The water-soluble dyes are, for example, beetroot juice, methylene blue and caramel.

The colorants may be present in an amount of from 0.5% to 30% by weight, relative to the total weight of the composition, preferably of from 3% to 20% by weight, and preferentially of from 5% to 15% by weight.

According to one particular embodiment of the invention, the emulsion may comprise at least one hydrophobic coated pulverulent material, in particular in an amount less than or equal to 20% by weight, relative to the total weight of the composition. Said pulverulent material may be selected from fillers, pulverulent colorants such as fillers, and pigments as described above. Said pulverulent material is coated with a hydrophobic agent as described above.

### Additives

The composition may contain other, usual cosmetic ingredients, which may be selected in particular from hydrophilic or lipophilic gellants and/or thickeners, antioxidants, fragrances, preservatives, neutralizers, sunscreen agents, vitamins, moisturizers, self-tanning compounds, active anti-wrinkle agents, emollients, hydrophilic or lipophilic active agents, free-radical scavenger agents, sequestrants, film-forming agents, and mixtures thereof.

A person skilled in the art will of course ensure that this or these optional further compounds, and/or their amount, is or are selected such that the advantageous properties of the composition according to the invention are not, or not substantially, adversely affected by the intended addition.

### Preparation of the Solid Emulsion

The composition according to the invention may be prepared by the following procedure:

Firstly, the mixture of the ingredients of the oily phase is prepared, by mixing and heating to a temperature of between 70° C. and 100° C. the waxes and the non-volatile oils, and then by adding with stirring, at a temperature of between 60° C. and 80° C., the volatile oils, and optionally the fillers and pigments.

Secondly, the mixture of the ingredients of the aqueous phase is prepared, comprising the water, surfactants and water-miscible solvents, by heating to a temperature of between 60° C. and 80° C.

The aqueous phase is then added to the oily phase, at a temperature of between 60° and 80° C., and the mixture is stirred with a turbine until the water-in-oil emulsion is obtained. The emulsion is then poured into a container, such as a dish, for example, after which it is cooled to ambient temperature to produce the solid emulsion.

### Hardness

In particular, the solid emulsion has a hardness such that the penetration force after 24 hours at 20° C. (counting in particular from the end of preparation of the solid emulsion) and at least one month at 37° C. is greater than or equal to 100 grams (g), being more particularly from 100 g to 1500 g.

The hardness of the solid emulsion is preferably from 200 g to 1000 g, more preferably from 300 g to 700 g.

The protocol for measuring the hardness of the emulsion is as follows:

At the end of preparation of the emulsion, the emulsion is poured hot into a dish and is kept at 20° C. for 24 hours. On the solid emulsion, a measurement is then made of the penetration force, by means of a texturometer sold under the name TA-XT21 by the company Rheo, equipped with the P/10 measuring spindle, with a diameter of 10 mm, the measurement conditions used being as follows:

Trigger force=1.0 g
Pre-speed=2.0 mm/s
Penetration speed=0.5 mm/s
Depth of penetration=2 mm

The penetration force, expressed in grams, is read off on the apparatus.

The solid emulsion is then stored at 37° C. for a month, after which it is placed at 20° C. for 24 hours before the hardness is measured (referred to as the hardness after one month at 37° C.), the conditions being the same as those described above.

The invention will now be illustrated by the following, non-limitative examples. Unless indicated otherwise, the values are expressed as percentages by weight relative to the total weight of said composition.

### EXAMPLES

### Examples 1 to 3

### Influence of the Type of Oil

| | | Example | Example | Example |
|---|---|---|---|---|
| | | 1 (inven- | 2 (com- | 3 (com- |
| | Ingredients | tive) | parative) | parative) |
| Fatty | POLYGLYCERYL-4 | 2.04 | 2.04 | 2.04 |
| phase | ISOSTEARATE | | | |
| | ISOLAN GI 34 from | | | |
| | GOLDSCHMIDT | | | |
| | POLYGLYCERYL-3 | 2.04 | 2.04 | 2.04 |
| | DIISOSTEARATE | | | |
| | LAMEFORM TGI | | | |
| | from COGNIS | | | |
| | PURIFIED | 4.08 | 4.08 | 4.08 |
| | CANDELILLA WAX | | | |
| | NC1630 from | | | |
| | CERA RICA NODA | | | |
| | PROPYLENE | 0.25 | 0.25 | 0.25 |
| | CARBONATE | | | |
| | Majority mixture | 16 | | |
| | of n-undecane and | | | |
| | n-tridecane, in | | | |
| | which the majority | | | |
| | compound in the | | | |
| | mixture is n- | | | |
| | undecane* | | | |
| | DICAPRYLYL | | 16 | |
| | CARBONATE (CETIOL | | | |
| | CC from Cognis) | | | |
| | PROPANEDIOL DI- | | | 16 |
| | CAPRYLATE (DUB | | | |
| | Zenoat from | | | |
| | STEARINERIES | | | |
| | DUBOIS) | | | |
| | DISTEARYLDI- | 0.41 | 0.41 | 0.41 |
| | METHYLAMMONIUM- | | | |
| | MODIFIED | | | |
| | HECTORITE | | | |
| | CAPRYLIC/CAPRIC | 13 | 13 | 13 |
| | ACID TRI- | | | |
| | GLYCERIDES | | | |
| | YELLOW IRON | 1.42 | 1.42 | 1.42 |
| | OXIDE COATED | | | |
| | WITH ALUMINIUM | | | |
| | STEAROYL | | | |
| | GLUTAMATE (3%) | | | |
| | RED IRON OXIDE | 0.78 | 0.78 | 0.78 |
| | COATED WITH | | | |
| | ALUMINIUM | | | |
| | STEAROYL | | | |
| | GLUTAMATE (3%) | | | |
| | BLACK IRON | 0.3 | 0.3 | 0.3 |
| | OXIDE COATED | | | |
| | WITH ALUMINIUM | | | |
| | STEAROYL | | | |
| | GLUTAMATE (3%) | | | |
| | ANATASE | 11.5 | 11.5 | 11.5 |
| | TITANIUM OXIDE | | | |
| | COATED WITH | | | |
| | ALUMINIUM | | | |
| | STEAROYL | | | |
| | GLUTAMATE | | | |
| | (97/3) | | | |
| | SERICITE | 10 | 10 | 10 |
| Aqueous | WATER | 30.43 | 30.43 | 30.43 |
| phase | GLYCEROL | 5 | 5 | 5 |
| | 1,3-BUTYLENE | 1.53 | 1.53 | 1.53 |
| | GLYCOL | | | |
| | OCTANE-1,2-DIOL | 0.51 | 0.51 | 0.51 |
| | SODIUM CHLORIDE | 0.71 | 0.71 | 0.71 |
| | | 100 | 100 | 100 |

*as prepared in accordance with patent application W02008/155059

Preparation Process:

The pigments are ground beforehand respectively in the undecane/tridecane mixture or the dicaprylyl carbonate or propanediol dicaprylate.

The fatty phase is then heated to 70° C. and is homogenized thoroughly with the pigments and the fillers.

The aqueous phase is also mixed and heated to 70° C., and then is added to the fatty phase for emulsification for 10 minutes.

Pouring takes place into dishes at ambient temperature, from a bulk product at 65° C.

The comparative examples result in a coarse emulsion which is unstable in manufacture. The castings do not give a uniform result.

### Examples 4 and 5

### Influence of the Type of Surfactant

| | | Example | Example |
|---|---|---|---|
| | | 4 (inven- | 5 (com- |
| | Ingredients | tive) | parative) |
| Fatty | PEG-30 DIPOLYHYDROXY- | | 4 |
| phase | STEARATE ARLACEL P135 | | |
| | from CRODA | | |
| | POLYGLYCERYL-3 DIISO- | 4 | |
| | STEARATE LAMEFORM TGI | | |
| | from COGNIS | | |
| | PURIFIED CANDELILLA WAX | 4.08 | 4.08 |
| | NC1630 from CERA RICA | | |
| | NODA | | |
| | PROPYLENE CARBONATE | 0.25 | 0.25 |
| | Majority mixture of n- | 16 | 16 |
| | undecane and n-tridecane, | | |
| | in which the n-undecane | | |
| | is the majority compound | | |
| | in the mixture* | | |
| | DISTEARYLDIMETHYL- | 0.41 | 0.41 |
| | AMMONIUM-MODIFIED | | |
| | HECTORITE | | |
| | ISOTRIDECYL ISONONANOATE | 13 | 13 |
| | YELLOW IRON OXIDE COATED | | |
| | WITH ALUMINIUM STEAROYL | 1.42 | 1.42 |
| | GLUTAMATE (3%) | | |
| | RED IRON OXIDE COATED | | |
| | WITH ALUMINIUM STEAROYL | 0.78 | 0.78 |
| | GLUTAMATE (3%) | | |
| | BLACK IRON OXIDE COATED | | |
| | WITH ALUMINIUM STEAROYL | 0.3 | 0.3 |
| | GLUTAMATE (3%) | | |
| | ANATASE TITANIUM OXIDE | | |
| | COATED WITH ALUMINIUM | 11.5 | 11.5 |
| | STEAROYL GLUTAMATE (3%) | | |
| | SERICITE | 10 | 10 |
| Aqueous | WATER | 29.82 | 29.82 |
| phase | GLYCEROL | 5 | 5 |
| | 1,3-BUTYLENE GLYCOL | 1.53 | 1.53 |
| | PHENOXYETHANOL | 0.5 | 0.5 |
| | METHYLPARABEN | 0.4 | 0.4 |
| | CHLORPHENESINE | 0.3 | 0.3 |
| | SODIUM CHLORIDE | 0.7 | 0.7 |
| | | 100 | 100 |

*as prepared according to patent application W02008/155059

Preparation Process

The pigments are ground beforehand in the undecane/tridecane mixture. The fatty phase is then heated to 70° C. and is homogenized thoroughly with the pigments and the fillers.

The aqueous phase is also mixed and heated to 70° C., and then is added to the fatty phase for emulsification for 10 minutes.

Pouring takes place into dishes at ambient temperature, from a bulk product at 65° C.

The comparative example leads to an emulsion which is a little less stable and exhibits no freshness effect on application.

### Example 6

### Fresh and Moisturizing Foundation

| | | Exam- |
|---|---|---|
| | Ingredients | ple 6 |
| | POLYGLYCERYL-3 DIISOSTEARATE | 4 |
| | LAMEFORM TGI from COGNIS | |
| | PURIFIED CANDELILLA WAX NC1630 from | 5 |
| | CERA RICA NODA | |
| | Majority mixture of n-undecane and n-tridecane, | 6 |
| | in which the n-undecane is the majority compound | |
| | in the mixture* | |
| | ISOPROPYL ISOSTEARATE | 10 |
| | CAPRYLIC/CAPRIC ACID TRIGLYCERIDES | 9.5 |
| | YELLOW IRON OXIDE COATED WITH | 1.42 |
| | ALUMINIUM STEAROYL GLUTAMATE (3%) | |
| | RED IRON OXIDE COATED WITH | 0.78 |
| | ALUMINIUM STEAROYL GLUTAMATE (3%) | |
| | BLACK IRON OXIDE COATED WITH | 0.3 |
| | ALUMINIUM STEAROYL GLUTAMATE (3%) | |
| | ANATASE TITANIUM OXIDE COATED WITH | 11.5 |
| | ALUMINIUM STEAROYL GLUTAMATE (3%) | |
| | NYLON 12 POWDER | 5 |
| | TALC | 3 |
| | SERICITE | 6 |
| Aqueous | WATER | 28.9 |
| phase | GLYCEROL | 5 |
| | OCTANE-1,2-DIOL | 1.5 |
| | PHENOXYETHANOL | 0.7 |
| | METHYLPARABEN | 0.4 |
| | CHLORPHENESINE | 0.3 |
| | SODIUM CHLORIDE | 0.7 |
| | | 100 |

*as prepared according to patent application W02008/155059

Preparation Process:

The pigments are ground beforehand in the undecane/tridecane mixture. The fatty phase is then heated to 70° C. and is homogenized thoroughly with the pigments and the fillers.

The aqueous phase is also mixed and heated to 70° C., and then is added to the fatty phase for emulsification for 10 minutes.

Pouring takes place into dishes at ambient temperature, from a bulk product at 60° C.

The foundation of example 6 exhibits a very good sensation of freshness on application. Its texture is fine and creamy, and it provides a moisturizing sensation after application and throughout the day.

### Example 7

### Compact and Moisturizing, Illuminating Care Product

| | | Exam- |
|---|---|---|
| | Ingredients | ple 7 |
| | POLYGLYCERYL-3 DIISOSTEARATE | 4 |
| | LAMEFORM TGI from COGNIS | |
| | PURIFIED CANDELILLA WAX NC1630 from | 5 |
| | CERA RICA NODA | |
| | Majority mixture of n-undecane and n-tridecane, | 6 |
| | in which the n-undecane is the majority compound | |
| | in the mixture* | |
| | ISOPROPYL ISOSTEARATE | 12.5 |
| | CAPRYLIC/CAPRIC ACID TRIGLYCERIDES | 12 |
| | MICA NACRE/TITANIUM DIOXIDE TIMIRON | 2 |
| | SILK BLUE from MERCK | |
| | NYLON 12 POWDER | 5 |
| | TALC | 3 |
| | SERICITE | 6 |
| Aqueous | WATER | 35.9 |
| phase | GLYCEROL | 5 |
| | OCTANE-1,2-DIOL | 1.5 |
| | PHENOXYETHANOL | 0.7 |
| | METHYLPARABEN | 0.4 |
| | CHLORPHENESINE | 0.3 |
| | SODIUM CHLORIDE | 0.7 |
| | | 100 |

*as prepared according to patent application W02008/155059

This compact product gives a fresh and moisturizing effect, and illuminates the skin.

### Example 8

### Fresh and Moisturizing Foundation

| | | Exam- |
|---|---|---|
| | Ingredients | ple 8 |
| | POLYGLYCERYL-3 DIISOSTEARATE | 4 |
| | LAMEFORM TGI from COGNIS | |
| | PURIFIED CANDELILLA WAX NC1630 from | 5 |
| | CERA RICA NODA | |
| | DODECANE (PARAFOL 12-97 from Sasol) | 6 |
| | ISOPROPYL ISOSTEARATE | 10 |
| | CAPRYLIC/CAPRIC ACID TRIGLYCERIDES | 9.5 |
| | YELLOW IRON OXIDE COATED WITH | 1.42 |
| | ALUMINIUM STEAROYL GLUTAMATE (3%) | |
| | RED IRON OXIDE COATED WITH | 0.78 |
| | ALUMINIUM STEAROYL GLUTAMATE (3%) | |
| | BLACK IRON OXIDE COATED WITH | 0.3 |
| | ALUMINIUM STEAROYL GLUTAMATE (3%) | |
| | ANATASE TITANIUM OXIDE COATED WITH | 11.5 |
| | ALUMINIUM STEAROYL GLUTAMATE (3%) | |
| | NYLON 12 POWDER | 5 |
| | TALC | 3 |
| | SERICITE | 6 |
| Aqueous | WATER | 28.9 |
| phase | GLYCEROL | 5 |
| | OCTANE-1,2-DIOL | 1.5 |
| | PHENOXYETHANOL | 0.7 |
| | METHYLPARABEN | 0.4 |
| | CHLORPHENESINE | 0.3 |
| | SODIUM CHLORIDE | 0.7 |
| | | 100 |

This composition is prepared by the protocol described in example 6.

This composition is pleasant and fresh on application.

### Examples 9 and 10

### Influence of the Type of Oils (Complementary Examples to Those Described in Examples 1 to 3 Above)

| | | Example | Example | Example |
|---|---|---|---|---|
| | | 6 (inven- | 9 (com- | 1 (com- |
| | Ingredients | tive) | parative) | parative) |
| Fatty | POLYGLYCERYL-3 | 4 | 4 | 4 |
| phase | DIISOSTEARATE | | | |
| | LAMEFORM TGI | | | |
| | from COGNIS | | | |
| | PURIFIED | 5 | 5 | 5 |
| | CANDELILLA WAX | | | |
| | NC1630 from CERA | | | |
| | RICA NODA | | | |
| | Majority mixture of | 6 | | |
| | n-undecane and n- | | | |
| | tridecane, in which | | | |
| | the n-undecane is | | | |
| | the majority compound | | | |
| | in the mixture* | | | |
| | Cyclopentasiloxane | | 6 | |
| | Isododecane | | | 6 |
| | ISOPROPYL | 10 | 10 | 10 |
| | ISOSTEARATE | | | |
| | CAPRYLIC/CAPRIC | 9.5 | 9.5 | 9.5 |
| | ACID TRI- | | | |
| | GLYCERIDES | | | |
| | YELLOW IRON | 1.42 | 1.42 | 1.42 |
| | OXIDE COATED | | | |
| | WITH ALUMINIUM | | | |
| | STEAROYL | | | |
| | GLUTAMATE (3%) | | | |
| | RED IRON OXIDE | 0.78 | 0.78 | 0.78 |
| | COATED WITH | | | |
| | ALUMINIUM | | | |
| | STEAROYL | | | |
| | GLUTAMATE (3%) | | | |
| | BLACK IRON OXIDE | 0.3 | 0.3 | 0.3 |
| | COATED WITH | | | |
| | ALUMINIUM | | | |
| | STEAROYL | | | |
| | GLUTAMATE (3%) | | | |
| | ANATASE TITANIUM | 11.5 | 11.5 | 11.5 |
| | OXIDE COATED | | | |
| | WITH ALUMINIUM | | | |
| | STEAROYL | | | |
| | GLUTAMATE (3%) | | | |
| | NYLON 12 POWDER | 5 | 5 | 5 |
| | TALC | 3 | 3 | 3 |
| | SERICITE | 6 | 6 | 6 |
| Aqueous | WATER | 28.9 | 28.9 | 28.9 |
| phase | GLYCEROL | 5 | 5 | 5 |
| | OCTANE-1,2-DIOL | 1.5 | 1.5 | 1.5 |
| | PHENOXYETHANOL | 0.7 | 0.7 | 0.7 |
| | METHYL PARABEN | 0.4 | 0.4 | 0.4 |
| | CHLOROPHENESINE | 0.3 | 0.3 | 0.3 |
| | SODIUM CHLORIDE | 0.7 | 0.7 | 0.7 |
| | | 100 | 100 | 100 |

*as prepared according to patent application W02008/155059

Preparation Process for Compositions 9 and 10, in Accordance with the Protocol Described in Example 6 Above:

The pigments are ground beforehand in the undecane/tridecane mixture or, respectively, in the cyclopentasiloxane or the isododecane. The fatty phase is then heated to 70° C. and is homogenized thoroughly with the pigments and the fillers.

The aqueous phase is also mixed and heated to 70° C., and then is added to the fatty phase for emulsification for 10 minutes.

Pouring takes place into dishes at ambient temperature, from a bulk product at 60° C.

Stability Study:

The macroscopic and microscopic appearance of each composition (quality of the emulsion) is evaluated 24 hours after its preparation and also after 2 months of storage.

Makeup Study:

The foundations are evaluated by a panel of experts on the criteria of freshness on application, lubricity, greasiness, stickiness, and also coverage, mattness and powder effect in the makeup result.

The foundation of example 6 (inventive) exhibits a very good sensation of freshness on application. Its texture is fine and creamy, and it provides a moisturizing sensation after application and throughout the day.

For the composition of Example 9 (comparative, with cyclopentasiloxane), the emulsion does not form correctly during preparation, and the appearance is adverse at 24 hours.

The composition of Example 10 (comparative) leads to a formula which is not very lubricious on application and is very sticky during and after application. This formula also has a microscopic appearance which is less satisfactory at 24 hours by comparison with that of Example 6.

All in all, these results confirm that the use of volatile linear alkane(s), especially C7-C14 alkane(s), in a solid water-in-oil emulsion comprising a non-silicone polyglycerolated surfactant and a polar wax, especially a natural or natural-origin wax, makes it possible to obtain a stable, solid emulsion which exhibits a fresh effect and enhanced cosmetic properties (lubricity, non-stickiness), by comparison with other hydrocarbon or silicone oils.

1. A solid water-in-oil emulsion comprising an aqueous phase emulsified in a fatty phase, and comprising:
a volatile linear alkane;
a non-silicone polyglycerolated surfactant; and
a polar wax.

2. The solid water-in-oil emulsion of claim 1, wherein the volatile linear alkane component comprises an alkane comprising from 7 to 14 carbon atoms.

3. The solid water-in-oil emulsion of claim 1, wherein the volatile linear alkane component is obtained by physically processing a plant.

4. The solid water-in-oil emulsion of claim 1, wherein the volatile linear alkane component comprises at least one selected from the group consisting of n-heptane, n-octane, n-nonane, n-undecane, n-dodecane, n-tridecane, and n-tetradecane.

5. The solid water-in-oil emulsion of claim 1, comprising at least two different volatile linear alkanes, which differ from one another by a carbon number n of at least 1.

6. The solid water-in-oil emulsion of claim 1, wherein the volatile linear alkane component is comprises a mixture of at least two volatile linear alkanes, comprising, relative to a total weight of alkanes in the mixture:
from 50% to 90% by weight of a Cn volatile linear alkane, wherein n is in a range from 7 to 14; and
from 10% to 50% by weight of a Cn+x volatile linear alkane, wherein x is greater than or equal to 1 and n+x is in a range from between 8 to 14.

7. The solid water-in-oil emulsion of claim 1, wherein the volatile linear alkane component comprises an n-undecane:n-tridecane (C11/C13) mixture comprising, relative to a total weight of alkanes in the mixture:
a. from 55% to 80% by weight of a C11 volatile linear alkane (n-undecane);
b. from 20% to 45% by weight of a C13 volatile linear alkane (n-tridecane).

8. The solid water-in-oil emulsion of claim 1, wherein the volatile linear alkane component comprises an alkane having a flash point of more than 60° C.

9. The solid water-in-oil emulsion of claim 1, wherein the non-silicone polyglycerolated surfactant has a formula selected from the group consisting of formulae RO[CH₂CH(CH₂OH)O]ₘH, RO[CH₂CH(OH)CH₂O]ₘH, and RO[CH(CH₂OH)CH₂O]ₘH,
wherein R is an alkyl group and m is an integer from 1 to 10.

10. The solid water-in-oil emulsion of claim 9, wherein the non-silicone polyglycerolated surfactant is at least one selected from the group consisting of polyglyceryl-4 isostearate and polyglyceryl-3 diisostearate.

11. The solid water-in-oil emulsion of claim 1, wherein the polar wax is at least one natural or natural-origin polar wax selected from the group consisting of beeswax, lanolin wax, rice bran wax, carnauba wax, candelilla wax, shellac wax, montan wax, orange wax, lemon wax, laurel wax, hydrogenated jojoba oil, and olive wax.

12. The solid water-in-oil emulsion of claim 1, comprising:
a) from 0.5% to 25% by weight of the volatile linear alkane, relative to the total weight of the emulsion;
b) from 0.5% to 6% by weight of the non-silicone polyglycerolated surfactant, relative to the total weight of the emulsion; and
c) 0.5% to 10% by weight of the polar wax, relative to the total weight of the emulsion.

13. The solid water-in-oil emulsion of claim 1, further comprising:
at least one filler selected from the group consisting of an inorganic filler and an organic filler.

14. The solid water-in-oil emulsion of claim 13, comprising at least one selected from the group consisting of a lamellar inorganic filler and a porous organic filler.

15. The solid water-in-oil emulsion of claim 1, further comprising;
a colorant.

16. A cosmetic makeup, comprising
the emulsion of claim 1.

17. A non-therapeutic method for treating a keratin material, the method comprising:
applying the emulsion of claim 1 to a keratin material.

18. The method of claim 17, wherein the keratin material is skin.
